(19) United States
(12) Patent Application Publication
(10) Pub. No.: US 2019/0059784 A1
(43) Pub. Date: Feb. 28, 2019

(54) PATIENT POSITIONING CHECK WITH MPRS AND CROSS-HAIR GRAPHICS

(71) Applicant: KONINKLIJKE PHILIPS N.V., EINDHOVEN (NL)

(72) Inventor: ERKKI TAPANI VAHALA, Hyvinkaa (FI)

(21) Appl. No.: 16/081,484

(22) PCT Filed: Mar. 3, 2017
(86) PCT No.: PCT/EP2017/054976

§ 371 Date: Aug. 31, 2018

(30) Foreign Application Priority Data
Mar. 8, 2016 (EP) 16159218.3

(51) Int. Cl. A61B 5/055; A61B 5/00

(52) U.S. Cl. CPC A61B 5/0555; A61B 5/743

(57) ABSTRACT

The invention relates to a method of operating an MR imaging system (10) with regard to positioning a subject of interest (20) to be imaged comprises steps of: —conducting (64) an MR scan, —displaying (66) at least one MR image obtained from the MR scan on a graphical user interface (28), and —displaying (68) at least one information (54, 56) in the at least one MR image indicating a position relative to the examination space (16). The invention further relates to an MR imaging system and a computer program implementing said method.

### FIELD OF THE INVENTION

The invention pertains to a method of operating a magnetic resonance (MR) imaging system with regard to positioning a subject of interest to be imaged, and an MR imaging system operated by employing such method.

### BACKGROUND OF THE INVENTION

In the current state of the art of MR imaging (MRI) and computer tomography (CT), it is known to employ external laser bridges with scanners for positioning a patient into a treatment position, onto a table top of a patient table prior to scanning. This convention comes from an established CT practice, where absolute patient positioning is used with lasers and tattoo-marks outside a scanner bore to replicate patient positioning to be repeated with linear accelerators (LINAC) for applying radiotherapy treatment. There, adjusting the patient position with the lasers comes as a natural by-product of laser usage. Typical positioning includes leveling of the patient hips to a horizontal plane and straightening of the spine, for instance perpendicular to a sagittal plane.

With MR and relative marking workflow, inclusion of lasers is less useful and can complicate the scanning procedure. The laser-based alignment is also decoupled from scanning, that is, the patient can move, for instance due to being startled by an unexpected patient tabletop movement, between the laser-based positioning and the scanning event, which leads to positioning errors.

For compensating a patient movement occurring between two consecutive scans, international application WO 2014006550 A2 proposes a magnetic resonance (MR) system and method that maintains geometric alignment of diagnostic scans during an examination of a patient. At least one processor is programmed to, in response to repositioning of the patient during the examination, perform an updated survey scan of the patient. A scan completed during the examination is selected as a template scan. A transformation map between the template scan and the updated survey scan is determined using a registration algorithm, and the transformation map is applied to a scan geometry of a remaining diagnostic scan of the examination. A scan plan for the remaining diagnostic scan is generated using the updated scan geometry. The remaining diagnostic scan is performed according to the scan plan.

### SUMMARY OF THE INVENTION

It is an object of the present invention to provide an improved method for ensuring correct positioning of a subject of interest to be imaged by a magnetic resonance (MR) imaging system, in particular by employing a bore-type MR imaging system.

In one aspect of the present invention, the object is achieved by a method of operating an MR imaging system with regard to positioning a subject of interest to be imaged. The MR imaging system comprises an examination space and a graphical user interface.

The method includes steps of:
conducting an MR scan,
displaying at least one MR image obtained from the MR scan on the graphical user interface, and
displaying at least one information in the at least one MR image indicating at least one out of a position and orientation relative to the examination space.

An advantage of the method lies in that it enables to check if the subject of interest has moved after entering the examination space of the MRI system, which can result in more reliable positioning of the subject of interest.

With large benefits, the invention is particularly applicable to a medical appliance in which an MR imaging system is combined with a radiotherapy apparatus for MR-guiding purposes. It can enable effective radiation therapy planning and successful and accurate radiation therapy dose rate delivery by carrying out magnetic resonance imaging in the exact treatment position.

The conducted MR scan may be a 3D survey scan. In principle, any MR scan that appears to suitable to the person skilled in the art may be conducted. More than one MR images obtained from the MR survey scan may be displayed on the graphical user interface.

Preferably, the method comprises a step of comparing at least one out of a desired positioning and a desired orientation of the subject of interest with the at least one displayed information. Deviations from the desired positioning and/or the desired orientation of the subject of interest can readily be obtained in this way.

Further also preferred, the method includes steps of:
based on a result of the step of comparing, deriving at least one corrective action for positioning the subject of interest on the patient table top towards at least one out of the desired position and the desired orientation, and
carrying out the at least one derived corrective action.

The desired positioning and/or the desired orientation of the subject of interest can quickly and reliably be obtained in this way. If, by chance, it turns out during the step of comparing that no corrective action is required at all, the remaining steps of the method are omitted.

Moreover, the method may include preparatory steps of:
arranging the subject of interest to be imaged on top of a patient table in accordance with a desired position and orientation relative to the patient table top,
positioning the patient table top within the examination space such that at least a portion of the subject of interest that is to be imaged is positioned within the examination space.

In a preferred embodiment of the method, the step of displaying at least one information comprises displaying a graphical information, and the step of comparing comprises comparing at least one out of a desired positioning and a desired orientation of the subject of interest with the at least one displayed graphical information.

In this way, the at least one corrective action for positioning the subject of interest on the patient table top towards at least one out of the desired position and the desired orientation can beneficially be expedited due to the high degree of comprehensibility of the provided graphical information.

Preferably, the step of displaying at least one information comprises displaying at least one straight line. By that, a large amount of information regarding at least one out of a position and orientation relative to the examination space can be conveyed to an operator with little effort.

Preferably, the step of displaying at least one information comprises displaying information with respect to a position of the magnetic isocenter.

Preferably, the information the position of the MR image relative to the examination space is provided by the MR imaging system itself.

The displaying in the at least one MR image may be carried out by employing an overlay or fade-in method. In principle, any method of displaying the at least one information in the at least one MR image that appears to be suitable to those skilled in the art may be employed.

Preferred, the at least one straight line is a horizontal straight line across the at least one displayed MR image. In this way, an at least one corrective action for positioning the subject of interest with regard to leveling can readily be obtained.

In some embodiments, the step of displaying at least one information comprises displaying two orthogonal straight lines. By that, corrective actions for positioning the subject of interest with regard to leveling and with regard to a lateral position can readily be obtained. One of the two orthogonal straight lines may be a horizontal straight line across the at least one displayed MR image.

Preferably, the at least one straight line crosses a position of the magnetic isocenter of a quasi-static magnetic field of the MR imaging system. The phrase “magnetic isocenter”, as used in this application, shall be understood particularly as the center point of the quasi-static magnetic field. By that, a fast, at-a-glance orientation of a relative position and orientation of the subject of interest with the examination space can be accomplished.

Usually, the magnetic isocenter defines an origin of a Cartesian coordinate system. In the case of a bore-type MR imaging system, the magnetic isocenter is usually arranged at the center of the bore.

Also preferably, the step of displaying at least one information comprises displaying grid lines. The term “grid lines”, as used in this application, shall be understood particularly as a set of lines that cross each other at right angles. In this way, an improved navigation within the at least one MR image can be facilitated. In particular, the grid lines may be arranged in an evenly spaced manner.

In another preferred embodiment, the method further comprises a step of manually adjusting the displayed graphical information to a desired position. In this way, a result of the at least one corrective action for positioning the subject of interest on the patient table top towards at least one out of the desired position and the desired orientation can readily be assessed.

In yet another preferred embodiment of the method,
the step of comparing at least one out of a desired positioning and a desired orientation of the subject of interest comprises applying an image analysis algorithm for segmentation to the at least one MR image, and
the step of displaying at least one information includes displaying information that represents at least one spatial relation between the at least one out of a desired positioning and a desired orientation of the subject of interest and a result of the step of applying an image analysis algorithm for segmentation.

Image segmentation algorithms are well known in the art, and are commercially available nowadays, e.g. as a software module within MATLAB®, and shall therefore not be described in more detail herein. The phrase “segmentation algorithm”, as used in this application, shall particularly encompass but shall not be limited to segmentation methods that are based on thresholding, clustering, compression, edge detection, and histogram methods. In principle, any segmentation algorithm that appears to be suitable to those skilled in the art may be employed.

By applying an image analysis algorithm for segmentation, the step of comparing yields positions of contours and landmarks relative to the examination space, which can beneficially be used for assessing a misalignment of the subject of interest regarding the desired positioning and/or orientation.

In particular, the step of displaying the at least one information may include displaying numerical information, by which the at least one corrective action can readily be expressed in a quantitative manner.

If a 3D scan is combined with applying an automatic segmentation algorithm to the at least one MR image, it is possible to automatically scale and plan the images to show organs, such as femur heads and spine that are particularly pertinent for detecting patient misalignment.

Preferably, the step of deriving at least one corrective action for positioning the subject of interest is followed by a further step of displaying information representing the at least one corrective action to the subject of interest to be imaged.

This is in particular beneficial if the subject of interest is able to follow instructions. If the subject of interest is not able to follow the instructions, the operator can move the subject of interest out of the examination space and can reposition manually, according to displayed information in the at least one MR image, for instance from in-room display.

Preferably, the step of displaying information representing the at least one corrective action to the subject of interest to be imaged includes displaying the desired position and/or orientation relative to the examination space, such that the subject of interest can autonomously take steps to carry out the at least one derived corrective action, being supported by displayed feedback information.

In particular, the displaying of the desired position and/or orientation relative to the examination space can be carried out by using display appliances, such as a projector.

A high degree of alignment between a momentary position and/or orientation of the subject of interest and the desired position and/or orientation relative to the examination space can be accomplished if at least the steps of:
conducting an MR scan,
   displaying at least one MR image obtained from the MR scan on the graphical user interface,
   displaying at least one information in the at least one MR image indicating at least one out of a position and orientation relative to the examination space,
   comparing at least one out of a desired positioning and a desired orientation of the subject of interest with the at least one displayed information,
   based on a result of the step of comparing, deriving at least one corrective action for positioning the subject of interest on the patient table top towards at least one out of the desired position and the desired orientation, and
   carrying out the at least one derived corrective action are repeated in an iterative manner.

It is another object of the invention to provide an MR imaging system configured for acquiring MR signals from at least a portion of a subject of interest and for providing MR images from the acquired MR signals. The MR imaging system comprises
an examination space provided to arrange at least the portion of the subject of interest within,
a main magnet that is configured for generating a quasi-static magnetic field B₀ at least in the examination space,
wherein the examination space is arranged within the magnetic field B₀ of the main magnet,
a control unit that is configured for controlling functions of the MR imaging system,
a signal processing unit provided for processing MR signals to generate at least one image of at least the portion of the subject of interest from the received MR signals, and
a graphical user interface.

The control unit and the image processing unit are configured to carry out steps of the method disclosed herein.

The phrase “being configured to”, as used in this application, shall in particular be understood as being specifically programmed, laid out, furnished or arranged.

The benefits presented in context with the method of operating an MR imaging system in accordance with the invention fully apply to the disclosed MR imaging system.

In a particularly preferable embodiment, the MR imaging system includes a radiotherapy apparatus. In this way, the presented benefits in context with the method of operating an MR imaging system in accordance with the invention can be achieved for MR-guided radiotherapy purposes. Particularly, the radiotherapy apparatus may be formed by a LINAC radiotherapy apparatus.

It is another aspect of the present invention to provide a software module for carrying out an embodiment of the disclosed method of operating an MR imaging system with regard to positioning a subject of interest to be imaged. The method steps to be conducted are converted into a program code of the software module. The program code is implementable in a digital data memory unit of an MR imaging system and is executable by a processor unit of the MR imaging system. The processor unit may be the processor unit of a control unit that is customary for controlling functions of the MR imaging system. The processor unit may, alternatively or supplementary, be another processor unit that is especially assigned to execute at least some of the method steps.

The software module can enable a robust and reliable execution of the method and can allow for a fast modification of method steps if required.

### BRIEF DESCRIPTION OF THE DRAWINGS

These and other aspects of the invention will be apparent from and elucidated with reference to the embodiments described hereinafter. Such embodiment does not necessarily represent the full scope of the invention, however, and reference is made therefore to the claims and herein for interpreting the scope of the invention.

In the drawings:

FIG. 1 shows a schematic illustration of a part of an embodiment of an MR examination system in accordance with the invention,

FIG. 2 illustrates contents of a graphical user interface of the MR examination system pursuant to FIG. 1 generated by applying an embodiment of the method in accordance with the invention,

FIG. 3 illustrates contents of the graphical user interface of the MR examination system pursuant to FIG. 1 generated by applying an alternative embodiment of the method in accordance with the invention,

FIG. 4 schematically illustrates graphical information displayed to the subject of interest, representing a corrective action,

FIG. 5 illustrates a flow chart of an embodiment of the method in accordance with the invention.

### DETAILED DESCRIPTION OF EMBODIMENTS

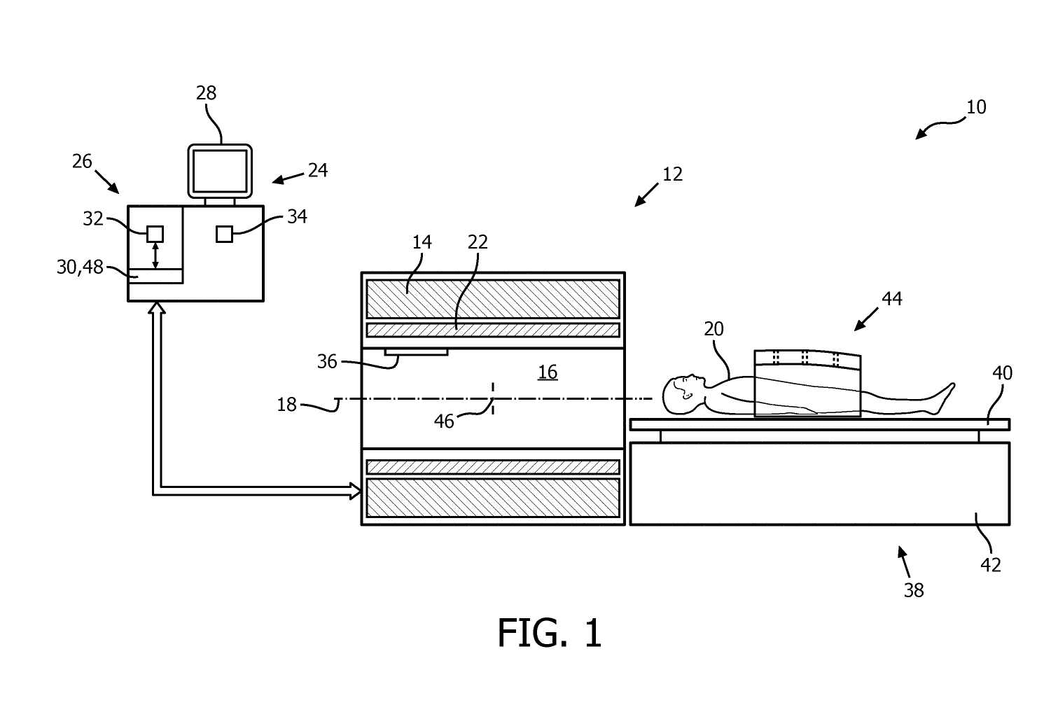

FIG. 1 shows a schematic illustration of an embodiment of a bore-type MR (MR) imaging system 10 in accordance with the invention. The MR imaging system 10 is configured for acquiring MR images from at least a portion of a subject of interest 20, usually a patient. The MR imaging system 10 comprises a scanner unit 12 including a main magnet 14. The main magnet 14 has a central bore that provides an examination space 16 around a center axis 18 for at least a portion of the subject of interest 20 to be positioned within, and is further configured for generating a quasi-static magnetic field B₀ at least in the examination space 16. The static magnetic field B₀ defines an axial direction usually denoted as the direction of the z-axis of a Cartesian coordinate system and being aligned in parallel to the center axis 18 of the examination space 16. A magnetic isocenter 46 of the quasi-static magnetic field B₀ is arranged on the center axis at a center of the bore, which coincides with an origin of the Cartesian coordinate system.

Although this specific embodiment is described as a bore-type magnetic resonance imaging system, it will readily be appreciated by those skilled in the art that the invention is also applicable to other types of magnetic resonance imaging systems, such as open (C-arm) MR imaging systems.

A customary patient table 38, in particular a radiotherapy simulation table, for supporting the subject of interest 20 includes a table support 42 and a table top 40 that is attached to the table support 42 in a slidable manner. The subject of interest 20, while being supported by the table top 40, can be transferred between positions within the examination space 16 and positions outside the examination space 16, as shown in FIG. 1.

Further, the MR imaging system 10 comprises a magnetic gradient coil system 22 configured for generating gradient magnetic fields superimposed to the static magnetic field B₀. The magnetic gradient coil system 22 is concentrically arranged within the bore of the main magnet 14 and comprises a plurality of coils to generate gradient magnetic fields in three dimensions, as is known in the art.

The MR imaging system 10 comprises a control unit 26 provided to control functions of the scanner unit 12, the magnetic gradient coil system 22, and other functions of the MR imaging system 10. The control unit 26 includes a processor unit 32, a digital data memory unit 30 and several human interface devices 24, including a graphical user interface 28 and a keyboard, provided for transferring information between the control unit 26 and an operator, usually a medical staff member.

Furthermore, the MR imaging system 10 includes an MR radio frequency antenna device 44 designed as a bridge-type MR radio frequency surface transmit/receive antenna that is configured for applying a radio frequency excitation field B₁ to nuclei of or within the subject of interest 20 for MR excitation during radio frequency transmit time periods to excite the nuclei of or within the subject of interest 20 for the purpose of MR imaging. To this end, the MR radio frequency antenna device 44 is connected to a radio frequency transmitter unit (not shown), and radio frequency power can be fed, controlled by the control unit 26, to the MR radio frequency antenna device 44.

The MR radio frequency antenna device 44 is further configured for receiving MR signals during radio frequency receive time periods from nuclei of or within the portion of the subject of interest 20 that have been excited by applying a radio frequency excitation field B₁. In an operational state of the MR imaging system 10, radio frequency transmit time periods and radio frequency receive time periods are taking place in a consecutive manner.

The radio frequency power of MR radio frequency is provided to the MR radio frequency antenna device 44 via a radio frequency switching unit during the radio frequency transmit time periods, as is known in the art. During the radio frequency receive time periods, the radio frequency switching unit, controlled by the control unit 26, directs the MR signals from the MR radio frequency antenna device 44 to a signal processing unit 34 that is configured for processing MR signals and for determining MR images of at least the portion of the subject of interest 20 from the acquired MR signals.

Moreover, the MR image system 10 includes an in-bore display appliance 36 for projecting information onto an inner surface of a bore wall lining (not shown).

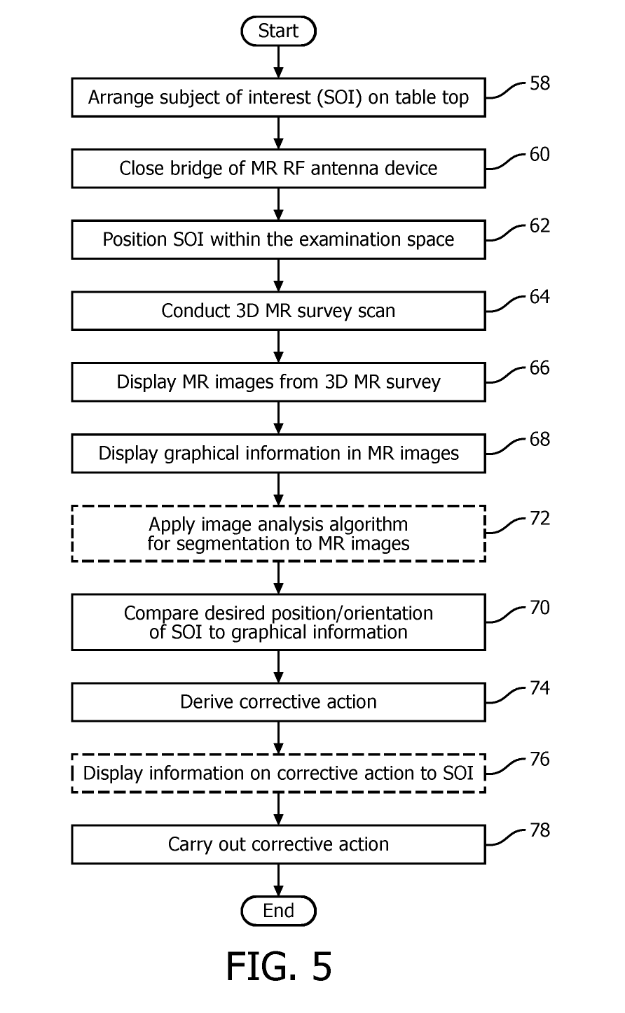

In the following, an embodiment of a method of operating the bore-type MR imaging system 10 with regard to positioning the subject of interest 20 to be imaged in accordance with the invention is described. A flow chart of the method is given in FIG. 5. It shall be understood that all involved units and devices are in an operational state and configured as illustrated in FIG. 1.

In order to be able to carry out parts of the method, the control unit 26 comprises a software module 48 (FIG. 1). The method steps to be conducted are converted into a program code of the software module 48, wherein the program code is implemented in the digital data memory unit 30 of the control unit 26 and is executable by the processor unit 32 of the control unit 26.

In a first step 58 of the method, the subject of interest 20 to be imaged is arranged on the table top 40 of the patient table 38 in accordance with the desired position and orientation relative to the patient table top 40. The step 58 of arranging the subject of interest 20 may be carried out using visual judgment of the operator. Alternatively or additionally, the step 58 of arranging the subject of interest 20 may be carried out by employing a commonly-used MR light-visor laser for determining the imaging isocenter. In this specific embodiment, the subject of interest 20 is arranged on the table top 40 in supine position, but principally other positions are as well contemplated.

Then, a bridge of the MR radio frequency antenna device 44 is closed and radio frequency MR coils are positioned on top of the coil bridge in another step 60.

In the next step 62, the operator positions the subject of interest 20 arranged on the table top 40 within the bore such that the subject of interest 20 is positioned within the examination space 16.

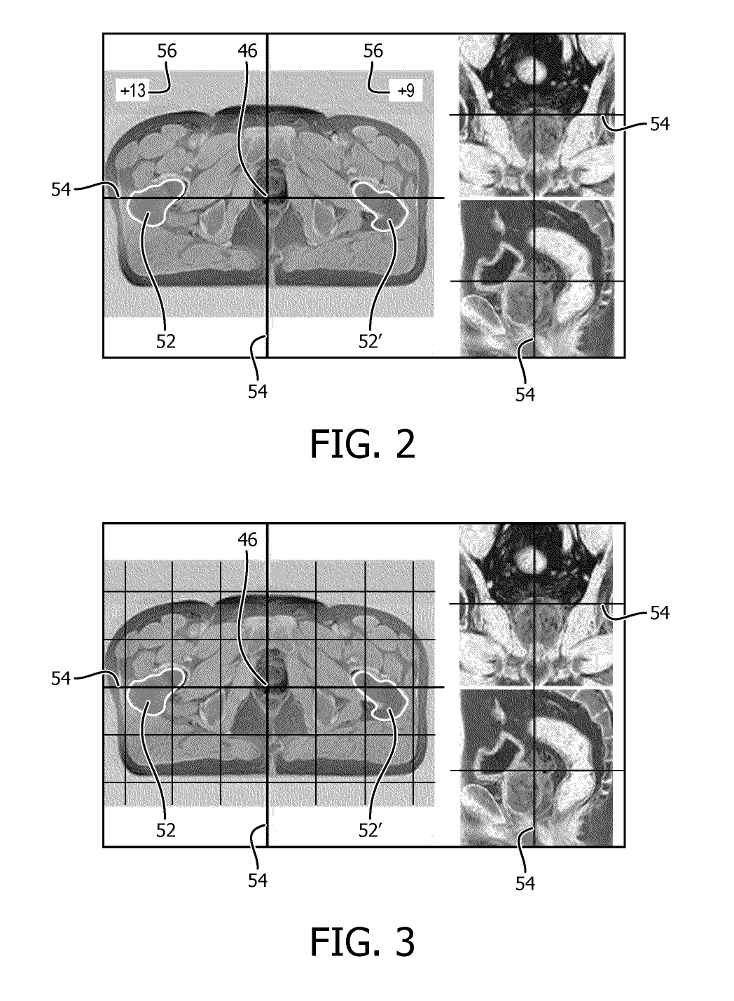

As a next step 64 of the method, a 3D MR survey scan is conducted, and three orthogonal MR images are generated, one each in the transverse plane, the sagittal plane and the coronal plane. The three MR images obtained from the MR survey scan are displayed in a next step 66 on the graphical user interface 28 as shown in FIG. 2.

By employing an overlay method, graphical information 54 is displayed in each one of the three orthogonal MR images in another step 68. In each one of the MR images, the graphical information 54 comprises two orthogonal straight lines across the respective MR image. Each one of the displayed straight lines crosses a position of the magnetic isocenter 46 of the quasi-static magnetic field B₀ and is arranged in parallel to one of the axes of the Cartesian coordinate system. The position and orientation of the straight lines relative to the examination space 16 is therefore known within tolerance margins that are typical in the art of manufacturing MR systems.

In an alternative embodiment, additional straight lines may be displayed, wherein each of the additional straight lines is arranged in parallel to one of the axes of the Cartesian coordinate system. The additional straight lines and the originally displayed straight line to which the additional straight lines are arranged in parallel may be spaced in an evenly manner so as to form grid lines, as is schematically shown in FIG. 3.

In a next step 70 of the method, a desired positioning and a desired orientation of the subject of interest 20 is compared to the displayed graphical information 54 by the operator. In the specific embodiment, the desired positioning is such that the anterior portion of each hip bone 52, 52′ of the subject of interest 20 shall be arranged to be aligned with the horizontal plane.

In this specific embodiment, in the step of comparing 70 it becomes obvious from the transverse plane MR image and the displayed straight line that the hip bones 52, 52′ of the subject of interest 20 are not arranged in the desired position, but are rather differently arranged relative to the horizontal plane. Based on a result of the step of comparing 70, the operator derives a corrective action for positioning the subject of interest 20 on the patient table top 40 towards the desired position and the desired orientation in another step 74.

In support of the step 70 of comparing and the step of deriving 74 the at least one corrective action, the operator can initiate an optional step 72 of applying an image analysis algorithm for segmentation to at least one of the MR images. In this specific embodiment, the image analysis algorithm is employed for delineating the hip bones 52, 52′ of the subject of interest 20, as shown in FIG. 2.

In case that the image analysis algorithm for segmentation has been applied, the step of displaying 68 at least one information also includes displaying numerical information 56 that represents a spatial relation between the desired positioning and the desired orientation of the subject of interest 20 and the result of the step 72 of applying an image analysis algorithm for segmentation. In the MR image of the transversal plane in FIG. 2, the numerical information 56 describes a distance of each one of the hip bones 52, 52′ to the horizontal plane that has to be compensated to reach the desired positioning in the transversal plane.

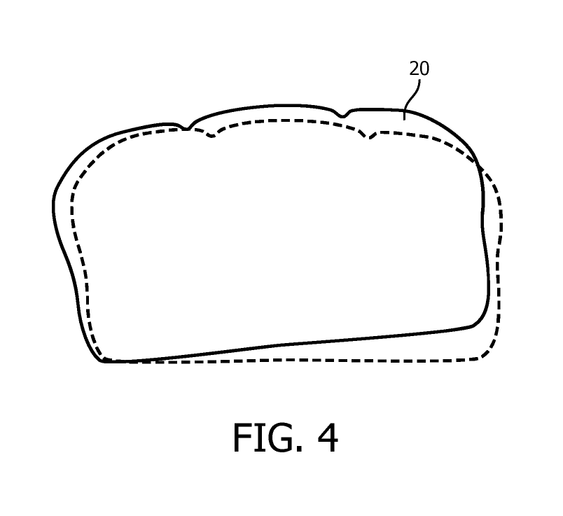

In the next step 78, the derived corrective action is carried out. To this end, the operator may communicate the derived corrective action to the subject of interest 20. Alternatively, the step 74 of deriving the at least one corrective action for positioning the subject of interest 20 is followed by a further step 76 of displaying information representing the at least one corrective action to the subject of interest 20 via the in-bore display appliance 36. FIG. 4 schematically illustrates the information displayed to the subject of interest 20, comprising an outline of the subject of interest 20 and the desired positioning and orientation in the transversal plane, indicating the corrective action to the subject of interest 20.

For enhanced precision of achieving the desired positioning and orientation, steps 64-78 can be executed in an iterative manner.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

REFERENCE SYMBOL LIST

| | |
|---|---|
| 10 | MR imaging system |
| 12 | scanner unit |
| 14 | main magnet |
| 16 | examination space |
| 18 | center axis |
| 20 | subject of interest |
| 22 | magnetic gradient coil system |
| 24 | human interface devices |
| 26 | control unit |
| 28 | graphical user interface |
| 30 | digital data memory unit |
| 32 | processor unit |
| 34 | signal processing unit |
| 36 | in-bore display appliance |
| 38 | patient table |
| 40 | table top |
| 42 | table support |
| 44 | MR radio frequency antenna device |
| 46 | magnetic isocenter |
| 48 | software module |
| 52 | hip bone |
| 54 | graphical information |
| 56 | numerical information steps |
| 58 | arrange subject of interest on table top |
| 60 | close MR RF antenna device bridge |
| 62 | position subject of interest |
| 64 | conduct 3D MR survey scan |
| 66 | display MR images on GUI |
| 68 | display information in MR image |
| 70 | compare desired positioning/orientation to graphical information |
| 72 | apply image analysis algorithm |
| 74 | derive corrective action |
| 76 | display corrective action information to subject of interest |
| 78 | carry out derived corrective action |

1. A method of operating a magnetic resonance (MR) imaging system with regard to positioning a subject of interest to be imaged, the MR imaging system including an examination space and a graphical user interface, wherein the method comprising:
displaying at least one MR image obtained from an MR scan on the graphical user interface, and
displaying at least one information in the at least one MR image indicating a position relative to the examination space; and
comparing a desired position and a desired orientation of the subject of interest with the at least one displayed information wherein the step of comparing a desired position and orientation of the subject of interest comprises applying an image analysis algorithm for segmentation to the at least one MR image, and the step of displaying at least one information includes displaying information that represents at least one spatial relation between a desired position and orientation of the subject of interest and a result of the step of applying an image analysis algorithm for segmentation.

2. (canceled)

3. The method of claim 1, further comprising:
based on a result of the step of comparing, deriving at least one corrective action for positioning the subject of interest on the patient table top towards at least one out of the desired position and the desired orientation, and
carrying out the at least one derived corrective action.

4. The method of claim 1, wherein the step of displaying at least one information comprises displaying graphical information and the step of comparing comprises comparing at least one out of a desired positioning and a desired orientation of the subject of interest with the at least one displayed graphical information.

5. The method of claim 1, wherein the step of displaying at least one information comprises displaying at least one straight line.

6. The method of claim 5, wherein the at least one straight line is a horizontal straight line across the at least one displayed MR image.

7. The method of claim 5, wherein the at least one straight line crosses a position of the magnetic isocenter of a quasi-static magnetic field of the MR imaging system.

8. The method of claim 1, wherein the step of displaying at least one information comprises displaying grid lines.

9. The method of claim 4, further comprising a step of manually adjusting the displayed graphical information to a desired position.

10. (canceled)

11. The method of claim 3, wherein the step of deriving at least one corrective action for positioning the subject of interest is followed by a further step of displaying information representing the at least one corrective action to the subject of interest to be imaged.

12. The method of claim 3, wherein at least the steps of:
conducting an MR scan,
is playing at least one MR image obtained from the MR scan on the graphical user interface,
displaying at least one information in the at least one MR image indicating at least one out of a position and orientation relative to the examination space,
comparing at least one out of a desired positioning and a desired orientation of the subject of interest with the at least one displayed information,
based on a result of the step of comparing, deriving at least one corrective action for positioning the subject of interest on the patient table top towards at least one out of the desired position and the desired orientation, and
carrying out the at least one derived corrective action are repeated in an iterative manner.

13. A magnetic resonance (MR) imaging system configured for acquiring MR signals from at least a portion of a subject of interest and for providing MR images from the acquired MR signals, the MR imaging system comprising:
an examination space provided to arrange at least the portion of the subject of interest within,
a main magnet that is configured for generating a quasi-static magnetic field B₀ at least in the examination space,
wherein the examination space is arranged within the magnetic field B₀ of the main magnet,
a control unit that is configured for controlling functions of the MR imaging system, and
a signal processing unit provided for processing MR signals to generate at least one image of at least the portion of the subject of interest from the received MR signals, and
a graphical user interface,
wherein the control unit and the signal processing unit are configured to carry out steps of the method as claimed in claim 1.

14. The MR imaging system as claimed in claim 13, further comprising a radiotherapy apparatus.

15. A software module for carrying out the method of claim 1, wherein the method steps to be carried out are converted into a program code of the software module, wherein the program code is implementable in a digital data memory unit of the MR imaging system and is executable by a processor unit of the MR imaging system.

16. The method of claim 1, the at least one information in the at least one MR image indicating the position relative to the examination space is the position relative to a magnetic isocenter of the MR imaging system.
